(19) United States
(12) Patent Application Publication
(10) Pub. No.: US 2005/0106227 A1
(43) Pub. Date: May 19, 2005

(54) Delivery of polymer conjugates of therapeutic peptides and proteins via coated microprojections

(76) Inventors: Samuel Zalipsky, Redwood City, CA (US); Johanna H. Bentz, Newark, CA (US)

Correspondence Address: Francis Law Group, 1942 Embarcadero, Oakland, CA, 94606, US

(21) Appl. No.: 10/972,231

(22) Filed: Oct. 21, 2004

Related U.S. Application Data
(60) Provisional application No. 60/515,398, filed on Oct. 28, 2003.

(51) Int. Cl. A61K 9/70; A61K 9/14

(52) U.S. Cl. 424/449

(57) ABSTRACT

An apparatus for transdermally delivering a biologically active agent to a patient comprising a microprojection member having a plurality of microprojections that are adapted to pierce the stratum comeum of the patient, the microprojection member having a biocompatible coating disposed thereon that includes a biologically active agent selected from the group consisting of peptide and protein conjugates.

### CROSS-REFERNCE TO RELATED APPLICATIONS

This application claims the benefit of U.S Provisional Application No. 60/515,398, filed Oct. 28, 2003.

### FIELD OF THE PRESENT INVENTION

The present invention relates generally to transdermal agent or drug delivery systems and methods. More particularly, the invention relates to a percutaneous agent delivery method and apparatus for delivery of polymer conjugates of therapeutic peptides and proteins.

### BACKGROUND OF THE INVENTION

Active agents (or drugs) are most conventionally administered either orally or by injection. Unfortunately, many agents are completely ineffective or have radically reduced efficacy when orally administered since they either are not absorbed or are adversely affected before entering the bloodstream and thus do not possess the desired activity. On the other hand, the direct injection of the agent into the bloodstream, while assuring no modification of the agent during administration, is a difficult, inconvenient, and uncomfortable procedure which sometimes results in poor patient compliance.

Hence, in principle, transdermal delivery provides for a method of administering active agents that would otherwise need to be delivered via hypodermic injection or intravenous infusion. Transdermal agent delivery offers improvements in both of these areas. Transdermal delivery, when compared to oral delivery, avoids the harsh environment of the digestive tract, bypasses gastrointestinal drug metabolism, reduces first-pass effects, and avoids the possible deactivation by digestive and liver enzymes.

The word “transdermal” is used herein as a generic term referring to passage of an active agent across the skin layers. The word “transdermal” refers to delivery of an agent (e.g., a therapeutic agent such as a drug or an immunologically active agent such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources including electricity (e.g., iontophoresis) and ultrasound (e.g., phonophoresis). While active agents do diffuse across both the stratum comeum and the epidermis, the rate of diffusion through the stratum comeum is often the limiting step. Many compounds, in order to achieve an effective dose, require higher delivery rates than can be achieved by simple passive transdermal diffusion. When compared to injections, transdermal agent delivery reduces or eliminates the associated pain and reduces the possibility of infection.

Theoretically, the transdermal route of agent administration could be advantageous for the delivery of many therapeutic proteins, since proteins are susceptible to gastrointestinal degradation and exhibit poor gastrointestinal uptake and transdermal devices are more acceptable to patients than injections. However, the transdermal flux of medically useful peptides and proteins is often insufficient to be therapeutically effective due to the relatively large size/molecular weight of these molecules. Often the delivery rate or flux is insufficient to produce the desired effect or the agent is degraded prior to reaching the target site, for example, while in the patient's bloodstream.

As is well known in the art, transdermal agent delivery systems generally rely on passive diffusion to administer the drug while active transdermal agent delivery systems rely on an external energy source, such as electricity, heat and ultrasound, to deliver the agent. Passive transdermal agent delivery systems, which are more common, typically include a drug reservoir containing a high concentration of active agent. The reservoir is adapted to contact the skin, which enables the active agent to diffuse through the skin and into the body tissues or bloodstream of a patient.

Transdermal agent flux is, in general, dependent upon the condition of the skin, the size and physical/chemical properties of the active agent molecule, and the concentration gradient across the skin. Because of the low permeability of the skin to many agents, transdermal delivery has had limited applications. This low permeability is attributed primarily to the stratum corneum, the outermost skin layer, which consists of flat, dead cells filled with keratin fibers (i.e., keratinocytes) surrounded by lipid bilayers. This highly-ordered structure of the lipid bilayers confers a relatively impermeable character to the stratum corneum.

One common method of enhancing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

A further method of enhancing transdermal agent flux is through the use of active transport systems. As stated, active transport systems use an external energy source to assist and, in many instances, enhance agent flux through the stratum corneum. One such enhancement for transdermal agent delivery is referred to as “electrotransport.” This mechanism uses an electrical potential, which results in the application of electric current to aid in the transport of the agent through a body surface, such as skin.

There also have been many techniques and systems developed to mechanically penetrate or disrupt the outermost skin layers thereby creating pathways into the skin in order to enhance the amount of agent being transdermally delivered. Early vaccination devices known as scarifiers generally include a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

However, a serious disadvantage in using a scarifier to deliver an active agent is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore, the tiny slits formed by the piercing elements heal quickly after removal of the device, thus limiting the passage of the liquid agent solution through the passageways created by the piercing elements and in turn limiting the transdermal flux of such devices.

Other systems and apparatus that employ tiny skin piercing elements to enhance transdermal drug delivery are disclosed in U.S. Pat. Nos. 5,879,326, 3,814,097, 5,279,54, 5,250,023, 3,964,482, Reissue U.S. Pat. No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed transdermal delivery systems further typically include a reservoir for holding the active agent and a delivery system to transfer the agent from the reservoir through the stratum corneum, such as by hollow tines of the device itself. One example of such a device is disclosed in PCT Pub. No. WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent to be delivered coated on the microprojections instead of contained in a physical reservoir. This eliminates the necessity of a separate physical reservoir and developing an agent formulation or composition specifically for the reservoir.

There are, however, several drawbacks and disadvantages associated with coated microprojection systems. As is know in the art, coated microprojection systems are generally limited in the amount of drug that can be coated and delivered, and depending on the size of the device and number of microprojections is typically limited to delivery of a few hundred micrograms of an active agent. There are additional drawbacks associated with coating microprojections (or arrays thereof) with several classes of active agents and formulations thereof, such as peptide and protein formulations.

As is known in the art, in order to efficiently coat the microprojection arrays, one must be able to prepare a stable, often highly concentrated, and sufficiently viscous solution of the polypeptide. For most polypeptides, these types of solutions are very difficult to achieve. Many polypeptides have limited solubility, or tend to precipitate from the solutions at pH values close to their pI or near physiological pH.

Typically, in order to increase viscosity or when high doses are required, the polypeptide concentration is increased and/or often, various additives, such as sugars and starches, are employed as viscosity enhancers as well as to retain stability of the polypeptide during coating and drying,. However, substantial amounts of sugars typically need to be added or the polypeptide concentration has to be high to substantially increase viscosity of an aqueous solution. Sugars also tend to dilute the peptide compared to the percent solids in the coating.

Therefore, in some instances, starches are employed. However, starches have the disadvantages that most starches are not approved for parental applications, are difficult to obtain in pure form and can adversely affect the stability of the polypeptide.

A high therapeutic dose of the polypeptide requires often unusually high concentrations of the polypeptide coating solution with a minimal content of excipients such as stabilizers and viscosity enhancers in order to reach a high percent of solid drug in the coating (see also 0021 above). Especially for high protein concentrations, and also when the polypeptide solution is exposed to shear and air-water interfaces during the coating process, both covalent and non-covalent aggregation and thus increased viscosity and precipitation often occur when preparing the polypeptide and/or protein coating solutions as well as during the coating process. However, it has been found that attachment of a water-soluble, biocompatible polymer, such as PEG, to proteins and peptides typically results in improved solubility, improved physical and chemical stability, lower aggregation tendency and enhanced flow characteristics (e.g., viscosity). Furthermore, PEG-proteins usually possess reduced immunogenicity, a very important attribute for a therapeutic protein formulation. The properties and applications of PEG-proteins were reviewed in J M Harris & S. Zalipsky (1997) Poly(ethylene glycol) chemistry and Biological Applications, ACS symposium Series 680, Washington, D. C.

Additionally, during and after the application of a microprojection array or patch, the coated polypeptides can, and in many instances will, undergo proteolytic degradation in the skin even before reaching the systemic circulation. It is believed that the proteolytic degradation is caused, in significant part, due to the presence of proteolytic enzymes produced by the skin cells. However, as discussed in detail herein, attachment of polymers to the polypeptides, such as PEG, will enhance the resistance to proteolysis. Further, it can be conceived that due to the improved solubility of the PEG attached polypeptide, solubility in the skin is improved and occurs more rapidly.

It is therefore an object of the present invention to provide a transdermal agent delivery apparatus and method that substantially reduces or eliminates the aforementioned drawbacks and disadvantages associated with prior art agent delivery systems.

It is another object of the present invention to provide a transdermal agent delivery apparatus and method for the delivery of polymer conjugates of therapeutic peptides and proteins.

It is another object of the present invention to provide a transdermal agent delivery apparatus having a coated microprojection array that delivers polymer conjugates of therapeutic peptides and proteins at an effective rate.

It is another object of the present invention to provide a transdermal agent delivery apparatus and method having an extended drug delivery profile.

### SUMMARY OF THE INVENTION

In accordance with the above objects and those that will be mentioned and will become apparent below, the apparatus for transdermally delivering a biologically active agent to a patient in accordance with this invention comprises a microprojection member having a plurality of microprojections that are adapted to pierce the stratum comeum of the patient, the microprojection member having a biocompatible coating having at least one biologically active agent disposed thereon, the biologically active agent being selected from the group consisting of peptide and protein conjugates.

Preferably, the peptide and protein conjugates with polymers are derived from the following biocompatible water-soluble polymers: polyethyleneglycol, polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers thereof, and polyethyleneoxide-polypropylene oxide.

Preferably, each of the microprojections has a length of less than 1000 microns, more preferably, less than 300 microns, even more preferably, less than 250 microns.

In a further embodiment of the invention, the biocompatible coating includes a vasoconstrictor. Preferably, the vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

The thickness of biocompatible coating disposed on the microprojections is preferably less than 50 microns. In one embodiment of the invention, the coating thickness is less than 25 microns.

The biocompatible coating provides a biologically effective amount of the biologically active agent or its polymer conjugate and, if employed, a biologically effective amount of the vasoconstrictor. The coating is further dried onto the microprojections using drying methods known in the art.

The biocompatible coating can be applied to and dried on the microprojections using known coating methods. For example, the microprojections can be immersed or partially immersed into an aqueous coating solution. Alternatively, the coating solution can be sprayed onto the microprojections. Preferably, the spray has a droplet size of about 10-200 picoliters. More preferably, the droplet size and placement is precisely controlled using printing techniques so that the coating solution is deposited directly onto the microprojections and not onto other “non-piercing” portions of the member having the microprojections.

The method for transdermally delivering a biologically active agent to a patient, in accordance with one embodiment of the invention, comprises the steps of (i) providing a microprojection member having a plurality of microprojections that are adapted to pierce the stratum comeum of the patient, (ii) coating the microprojection member with a biocompatible coating having at least one biologically active agent, the biologically active agent being selected from the group consisting of peptide and protein conjugates and (iii) applying the microprojection member to the skin of the patient, whereby the microprojection members pierce the stratum corneum of the patient and deliver the biologically active agent.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection array;

FIG. 2 is a perspective view of the microprojection array shown in FIG. 1 having a coating deposited on the microprojections, according to the invention;

FIG. 2A is a cross-sectional view of a single microprojection taken along line 2A-2A in FIG. 2, according to the invention;

FIG. 3 is a plane view of a skin proximal side of a microprojection array, illustrating the division of the array into various drug delivery segments, according to the invention;

FIG. 4 is a side sectional view of a further embodiment of a microprojection array having different coatings applied to different microprojections, according to the invention;

FIG. 5 is a side sectional view of a microprojection array having an adhesive backing;

FIG. 6 is a side sectional view of a retainer having a microprojection member disposed therein; and

FIG. 7 is a perspective view of the retainer shown in FIG. 7.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and substantially concurrent with transdermal flux of the agent, during transdermal flux of the agent, during and after transdermal flux of the agent, and/or after transdermal flux of the agent. Additionally, two or more biologically active agents may be coated onto the microprojections resulting in co-delivery of the biologically active agents.

The term “biologically active agent”, as used herein, refers to a composition of matter or mixture containing a drug which is pharmacologically effective when administered in a therapeutically effective amount. Examples of such active agents include, without limitation, polymer conjugates of therapeutic peptides or proteins, Preferred polymers conjugated to the polypeptide include polyethyleneglycol, polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers thereof, and polyethyleneoxide-polypropylene oxide.

It is to be understood that more than one biologically active agent may be incorporated into the coatings of this invention and that the use of the term “active agent” in no way excludes the use of two or more such active agents.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed in the coatings of the invention will be that amount necessary to deliver a therapeutically effective amount of the active agent to achieve the desired therapeutic result. In practice, this will vary widely depending upon the particular pharmacologically active agent being delivered, the site of delivery, the severity of the condition being treated, the desired therapeutic effect and the dissolution and release kinetics for delivery of the agent from the coating into skin tissues.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed in the coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The terms “microprojections” and “microprotrusions”, as used herein, refer to piercing elements that are adapted to pierce or cut through the stratum comeum into the underlying epidermis layer, or epidermis and dermis layers, of the skin of a living animal, particularly a mammal and more particularly a human.

In one embodiment of the invention, the microprojections have a projection length less than 1000 microns. In a further embodiment, the microprojections have a projection length of less than 300 microns, more preferably, less than 250 microns. The microprojections typically have a width and thickness of about 5 to 50 microns. The microprojections may be formed in different shapes, such as needles, hollow needles, blades, pins, punches, and combinations thereof.

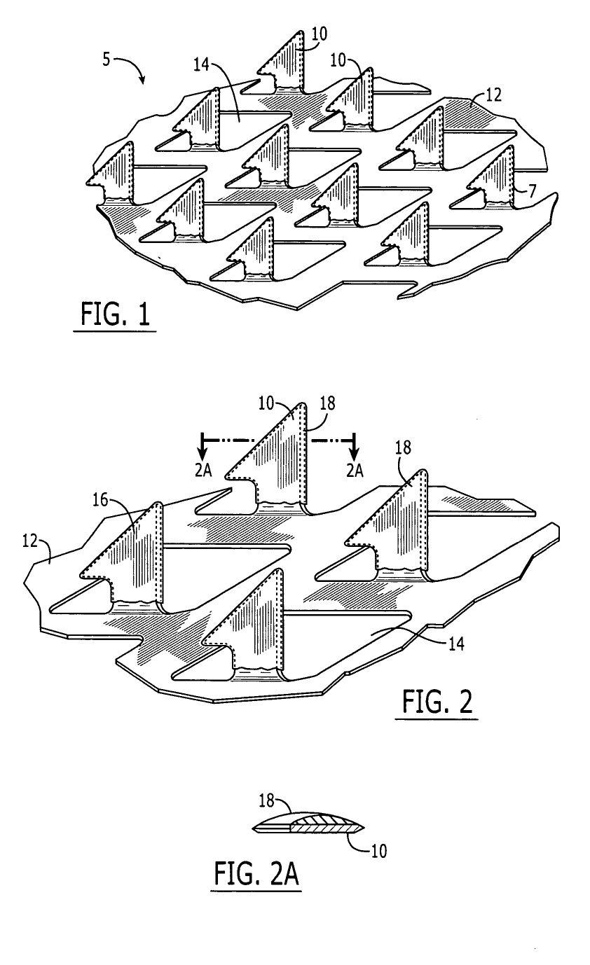

The term “microprojection array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as that shown in FIG. 1. The microprojection array may also be formed in other known manners, such as by forming one or more strips having microprojections along an edge of each of the strip(s) as disclosed in U.S. Pat. No. 6,050,988.

References to the area of the sheet or member and reference to some property per area of the sheet or member are referring to the area bounded by the outer circumference or border of the sheet.

The term “solution” shall include not only compositions of fully dissolved components but also suspensions of components including, but not limited to, protein virus particles, inactive viruses, and split-virions.

The term “pattern coating”, as used herein, refers to coating an active agent onto selected areas of the microprojections. More than one biologically active agent can be pattern coated onto a single microprojection array. Pattern coatings can be applied to the microprojections using known micro-fluid dispensing techniques such as micropipeting and ink jet coating.

As indicated above, the present invention comprises an apparatus and system for extended transdermal delivery of biologically active agents, particularly, polymer conjugates of therapeutic peptides and proteins. The system generally includes a microprojection member having a microprojection array comprising a plurality of microprojections that are adapted to pierce through the stratum corneum into the underlying epidermis layer, or epidermis and dermis layers.

Preferably, the microprojections have a coating thereon that contains at least one biologically active agent. Upon piercing the stratum corneum layer of the skin, the agent-containing coating is dissolved by body fluid (intracellular fluids and extracellular fluids such as interstitial fluid) and released into the skin for local or systemic therapy.

According to the invention, the kinetics of the coating dissolution and release will depend on many factors including the nature of the biologically active agent, the coating process, the coating thickness and the coating composition (e.g., the presence of coating formulation additives). Depending on the release kinetics profile, it may be necessary to maintain the coated microprojections in piercing relation with the skin for extended periods of time (e.g., up to about 8 hours). This can be accomplished by anchoring the microprojection member to the skin using adhesives or by using anchored microprojections, such as described in WO 97/48440, which is incorporated by reference herein in its entirety.

Referring now to FIG. 1, there is shown one embodiment of a microprojection member 5 for use with the present invention. As illustrated in FIG. 1, the microprojection member 5 includes a microprojection array 7 having a plurality of microprojections 10. The microprojections 10 preferably extend at substantially a 90° angle from the sheet 12, which includes openings 14.

According to the invention, the sheet 12 may be incorporated into a delivery patch, including a backing 15 for the sheet 12, and may additionally include adhesive for adhering the patch to the skin (see FIG. 5). In this embodiment, the microprojections 10 are formed by etching or punching a plurality of microprojections 10 from a thin metal sheet 12 and bending the microprojections 10 out of the plane of the sheet 12.

The microprojection member 5 can be manufactured from various metals, such as stainless steel, titanium, nickel titanium alloys, or similar biocompatible materials, such as polymeric materials. Preferably, the microprojection member 5 is manufactured out of titanium.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

Referring now to FIG. 2, there is shown the microprojection member 5 having microprojections 10 that include an agent-containing, biocompatible coating 16. According to the invention, the coating 16 can partially or completely cover each microprojection 10. For example, the coating 16 can be in a dry pattern coating on the microprojections 10. The coating 16 can also be applied before or after the microprojections 10 are formed.

According to the invention, the coating 16 can be applied to the microprojections 10 by a variety of known methods. Preferably, the coating is only applied to those portions the microprojection member 5 or microprojections 10 that pierce the skin (e.g., tips 18).

One such coating method comprises dip-coating. Dip-coating can be described as a means to coat the microprojections by partially or totally immersing the microprojections 10 into a coating solution. By use of a partial immersion technique, it is possible to limit the coating 16 to only the tips 18 of the microprojections 10.

A further coating method comprises roller coating, which employs a roller coating mechanism that similarly limits the coating 16 to the tips 18 of the microprojections 10. The roller coating method is disclosed in U.S. application Ser. No. 10/099,604, which is incorporated by reference herein in its entirety.

As discussed in detail in the noted application, the disclosed roller coating method provides a smooth coating that is not easily dislodged from the microprojections 10 during skin piercing. The smooth cross-section of the microprojection tip coating is Further illustrated in FIG. 2A.

According to the invention, the microprojections 10 can further include means adapted to receive and/or enhance the volume of the coating 16, such as apertures (not shown), grooves (not shown), surface irregularities (not shown) or similar modifications, wherein the means provides increased surface area upon which a greater amount of coating can be deposited.

Another coating method that can be employed within the scope of the present invention comprises spray coating. According to the invention, spray coating can encompass formation of an aerosol suspension of the coating composition. In a preferred embodiment, an aerosol suspension having a droplet size of about 10 to 200 picoliters is sprayed onto the microprojections 10 and then dried.

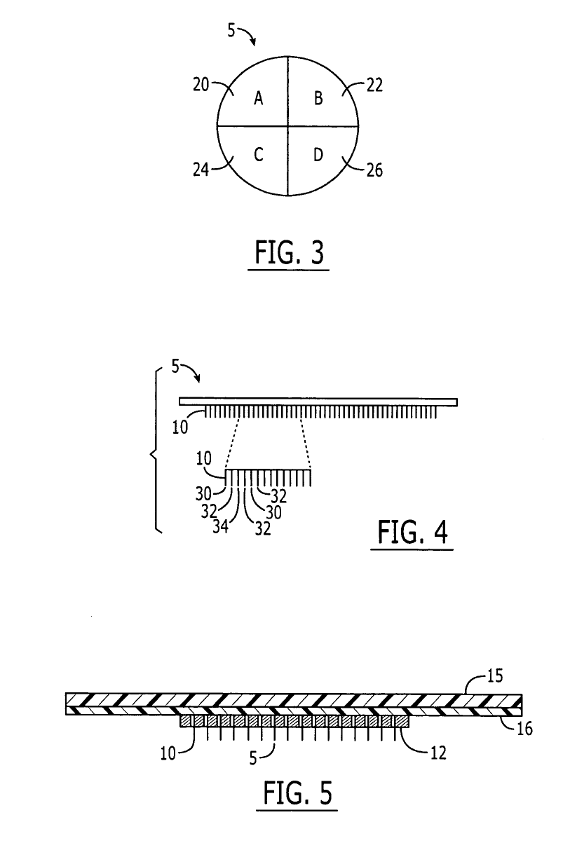

Referring now to FIG. 3, in a further embodiment of the invention, a different coating is applied to different segments of the microprojection member 5, designated 20-26. As will be appreciated by one having ordinary skill in the art, the noted arrangement allows a single microprojection array 7 to be employed to delivery more than one biologically active agent during use.

Referring now to FIG. 4, in another embodiment, a very small quantity of the coating solution is deposited onto the microprojections 10 via pattern coating. As illustrated in FIG. 4, each of the microprojections 10 can further be coated with a different biocompatible coating (designated generally 30-36).

The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In one embodiment of the invention, the coating solution formulations applied to the microprojection member to form solid coatings comprise liquid compositions (or coating solutions) having a biocompatible carrier and at least one biologically active agent. The biocompatible carrier can include, without limitation, human albumin, polyglutamic acid, polyaspartic acid, polyhistidine, pentosan polysulfate and polyamino acids. According to the invention, the active agent can be dissolved within the biocompatible carrier or suspended within the carrier.

The concentration of the biologically active agent in the coating solution is preferably less than approximately 40 wt. %, more preferably, in the range of approximately 2-20 wt. %.

According to the invention, the concentration of the biologically active agent in the solid coating(s) can be up to approximately 95 wt. %. In one embodiment, the concentration of the biologically active agent in the solid coating(s) is thus in the range of approximately 5-80 wt. %.

Preferably, the coating solution has a viscosity less than approximately 500 centipoise and greater than 3 centipoise in order to effectively coat each microprojection 10. More preferably, the coating solution has a viscosity in the range of approximately 10-100 centipoise.

According to the invention, the desired coating thickness is dependent upon the density of the microprojections per unit area of the sheet and the viscosity and concentration of the coating composition as well as the coating method chosen. Also, the coating thickness is limited as for it not to hinder penetration or piercing through the skin. Preferably, the coating thickness is less than 50 microns, more preferably, less than 25 microns.

In one embodiment, the coating thickness is less than 50 microns, more preferably, less than 10 microns as measured from the microprojection surface. Even more preferably, the coating thickness is in the range of approximately 1 to 10 microns.

According to the invention, the total amount of the biologically active agent coated on the microprojections of a microprojection array can be in the range of 1 microgram to 1 milligram. Amounts within this range can be coated onto a microprojection array of the type shown in FIG. 1 having an area of up to 10 cm² and a microprojection density of up to 2000 microprojections per cm².

In one embodiment of the invention, the amount of biologically active agent delivered to a patient from a 1 cm² microprojection array is in the range of approximately 5-75 μg.

As indicated above, the coatings of the invention comprise at least one biologically active agent. Preferably, the biologically active agent comprises a polymer conjugate of therapeutic peptides and proteins. More preferably, the biologically active agent is conjugated with at least one of the following biocompatible polymers: polyethyleneglycol, polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers thereof, and polyethyleneoxide-polypropylene oxide.

Applicants have found that the use of PEGylated proteins instead of unmodified, native, proteins provides many advantages, including (i) extension of activity duration in vivo, (ii) reduction of immunogencity and antigenicity, (iii) reduction in aggregate formation, (iv) increased resistance to proteolytic degradation, (v) improved physical and chemical stability, such as during the coating and drying process and upon storage and (vi) significantly improved solubility and ability to form stable concentrated solutions that facilitate efficient coating of microprojections.

Applicants have further found that PEGylated proteins have high solubility in physiologic solutions and near neutral pH. PEGylated proteins also facilitate solubility in the dermis from a coated solid state.

According to the invention, the coatings of the invention can include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

The coatings of the invention can further include a vasoconstrictor to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

Other formulation additives, such as stabilizers and excipients known in the art, can also be added to the coating solution as long as they do not adversely affect the necessary solubility and viscosity characteristics of the coating solution and the physical integrity of the dried coating.

In all cases, after a coating has been applied, the coating solution is dried onto the microprojections 10 by various means. In a preferred embodiment of the invention, the coated member 5 is dried in ambient room conditions. However, various temperatures and humidity levels can be used to dry the coating solution onto the microprojections. Additionally, the coated member 5 can be heated, lyophilized, freeze dried or similar techniques used to remove the water from the coating.

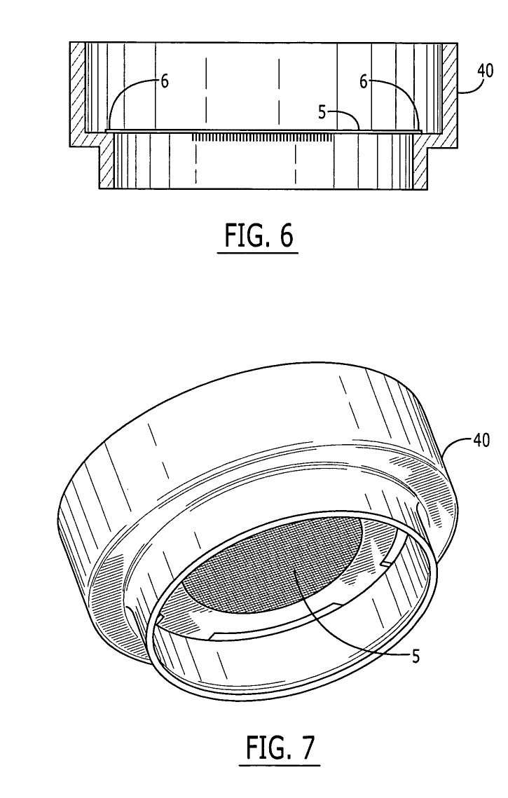

Referring now to FIGS. 6 and 7, for storage and application, the microprojection member 5 is preferably suspended in a retainer ring 40 by adhesive tabs 6, as described in detail in Co-Pending U.S. application Ser. No. 09/976,762 (Pub. No. 2002/0091357), which is incorporated by reference herein in its entirety.

After placement of the microprojection member 5 in the retainer ring 40, the microprojection member 5 is applied to the patient's skin. Preferably, the microprojection member 5 is applied to the skin using an impact applicator, such as disclosed in Co-Pending U.S. application Ser. No. 09/976,798, which is incorporated by reference herein in its entirety.

As will be appreciated by one having ordinary skill in the art, the present invention can also be employed with the transdermal drug delivery system and apparatus disclosed in Co-Pending Application No. 60/514,433.

As will also be appreciated by one having ordinary skill in the art, the present invention can similarly be employed in conjunction with a wide variety of electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., an agent or agent precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophotesis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported.

From the foregoing description, one of ordinary skill in the art can easily ascertain that the present invention, among other things, provides an effective and efficient means for extending the transdermal delivery of biologically active agents to a patient.

As will be appreciated by one having ordinary skill in the art, the present invention provides many advantages, such as:
Transdermal delivery of polymer conjugates of therapeutic peptides and proteins
Extended delivery profiles of biologically active agents.

The use of PEGylated proteins instead of unmodified, native, proteins provides many additional advantages, including:
Extension of activity duration in vivo
Reduction of immunogencity and antigenicity
Reduction in aggregate formation
Increased resistance to proteolytic degradation
Improved solubility for coating
Improved solubility in the skin and uptake into circulation
Improved physical and chemical stability in solution and solid state
Improved ability to form highly concentrated (above 2%, and typically between 5-25%) polypeptide solutions that facilitate efficient coating of microprojections
Protection during the coating process (e.g.: shear, air-water interfaces)

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. An apparatus for transdermally delivering a biologically active agent to a patient comprising a microprojection member including a plurality of stratum corneum-piercing microprojections having a biocompatible coating disposed thereon, said biocompatible coating including a biologically active agent selected from the group consisting of peptide and protein conjugates.

2. The apparatus of claim 1, wherein said peptide and protein are conjugated to a polymer selected from the group consisting of polyethyleneglycol, polyvinylpyrrolidone, polyvinylmethylether, polymethyloxazoline, polyethyloxazoline, polyhydroxypropyloxazoline, polyhydroxypropyl-methacrylamide, polymethacrylamide, polydinethyl-acrylamide, polyhydroxypropylmethacrylate, polyhydroxyethylacrylate, hydroxymethylcellulose, hydroxyethylcellulose, polyethyleneglycol, polyaspartamide, copolymers thereof, and polyethyleneoxide-polypropylene oxide.

3. The apparatus of claim 1, wherein each of said microprojections has a length of less than 1000 microns.

4. The apparatus of claim 3, wherein each of said microprojections has a length of less than 300 microns.

5. The apparatus of claim 4, wherein each of said microprojections has a length less than 250 microns.

6. The apparatus of claim 1, wherein said biocompatible coating further comprises a vasoconstrictor.

7. The apparatus of claim 1, wherein said biocompatible coating has a thickness less than approximately 50 microns.

8. The apparatus of claim 1, wherein said biocompatible coating has a thickness less than approximately 25 microns.

9. The apparatus of claim 1, further comprising an applicator having a contacting surface, 1 wherein said microprojection member is releasably mounted on said applicator by a retainer and wherein said applicator, once activated, brings said contacting surface into contact with said microprojection member in such a manner that said microprojection member can strike a stratum comeum of a patient with a power of at least 0.05 joules per cm² of microprojection member in 10 milliseconds or less.

10. A method for transdermally delivering a biologically active agent to a patient, comprising the steps of:
providing a microprojection member having a plurality of microprojections that are adapted to pierce said patient's stratum comeum;
coating said microprojection member with a coating formulation having said biologically active agent to form a biocompatible coating, wherein said biologically active agent is selected from the group consisting of peptide and protein conjugates; and
applying said microprojection member to said patient's skin, whereby said microprojection members pierce said stratum comeum and deliver said biologically active agent.

11. The method of claim 10, wherein said step of coating said microprojection member comprises immersing said microprojections in said coating formulation.

12. The method of claim 10, wherein said step of coating said microprojection member comprises spraying said coating formulation onto said microprojections.

13. The method of claim 10, further comprising the steps of:
providing an applicator having a contacting surface, wherein said microprojection member is releasably mounted on said applicator by a retainer; and activating said applicator to bring said contacting surface into contact with said microprojection member in such a manner that said microprojection member strikes said stratum comeum.
